(19) United States
(12) Patent Application Publication
(10) Pub. No.: US 2022/0119889 A1
(43) Pub. Date: Apr. 21, 2022

(54) PROGNOSTIC MARKERS IN LUNG CANCER, PROGNOSTIC TYPING MODEL OF LUNG CANCER, AND APPLICATION THEREOF

(71) Applicant: Lisen Imprinting Diagnostics Wuxi Co., Ltd., Wuxi, Jiangsu Province (CN)

(72) Inventors: Tong Cheng, Wuxi (CN); Ning Zhou, Wuxi (CN)

(21) Appl. No.: 17/295,644

(22) PCT Filed: Nov. 21, 2019
(86) PCT No.: PCT/CN2019/119966

§ 371 Date: Jul. 20, 2021

(30) Foreign Application Priority Data
Nov. 21, 2018 (CN) 201811391555.8
Nov. 20, 2019 (CN) 201911140625.7

(51) Int. Cl. C12Q 1/6886; C12Q 1/6841; A61K 31/7105; A61K 38/46

(52) U.S. Cl. CPC C12Q 1/6886; C12Q 1/6841; A61K 31/7105; C12Q 2600/136; C12Q 2600/118; C12Q 2600/158; A61K 38/465

(57) ABSTRACT

The present application provides a prognostic marker for lung cancer, a prognostic typing model for lung cancer, and applications thereof. The marker includes imprinted genes Dcn, Peg10, Snrpn/Snurf, and Trappc9. The four imprinted genes Dcn, Peg10, Snrpn/Snurf, and Trappc9 used in the present application have a significant correlation with lung cancer prognosis. Imprinted gene Dcn is the most sensitive and most specific marker for lung cancer prognosis and can serve an indicative purpose earlier than clinicopathologic features. The product of a total expressed quantity of each of the four imprinted genes and an expressed quantity of the imprinted gene with a copy number variation contributes to creating the prognostic typing model for lung cancer, thereby enabling the prediction of the five-year survival rates of individual lung cancer patients and the provision of accurate, useful prognosis information to lung cancer patients.

### TECHNICAL FIELD

The present application relates to the field of biotechnology; to the field of genetic diagnosis; to a prognostic marker for a tumor, a prognostic typing model for the tumor, and applications thereof; and more particularly to a prognostic marker for lung cancer, a prognostic typing model for lung cancer, and applications thereof.

### INCORPORATION OF SEQUENCE LISTING

This application includes a Sequence Listing which is being submitted in ASCII format via EFS-Web, named “LIS008US_ST25.txt,” which is 1 KB in size and created on Nov. 12, 2021. The contents of the Sequence Listing are incorporated herein by reference in their entirety.

### DESCRIPTION OF RELATED ART

Lung cancer, or pulmonary carcinoma, is the malignant tumor with the highest morbidity and mortality in the world. According to statistics of the World Health Organization (WHO), or more specifically World Cancer Report 2014, the year 2012 saw 1.82 million new cases of lung cancer worldwide, including 1.59 million deaths. In China, the same year saw 733 thousand newly diagnosed cases of lung cancer, including 610 thousand deaths, with lung cancer having the highest male morbidity and male mortality, the second highest female morbidity, and the highest female mortality of all cancers, according to the same report. The survival rate of a patient with lung cancer is closely related to the progression of the cancer. Patients with stage I lung cancer have a five-year survival rate as high as 70%-90%, whereas patients with stage IV lung cancer have a five-year survival rate of 10% at most. Early diagnosis and early treatment, therefore, are critical to saving the lives of patients with lung cancer. Currently, therapy targeted at specific driver gene mutations has relatively good results, but more than 60% of lung cancer patients do not have specific gene mutations that can serve as drug targets. In those cases, all that can be done is to keep trying different chemotherapy drugs, observe the treatment results, and then decide whether to continue with the current treatment or change the current drug. Some patients whose lung cancer develops relatively fast may therefore miss their best timing for treatment because an effective chemotherapy drug is not used in time. Accordingly, a molecular marker capable of predicting the prognosis of a patient in an early cancer stage is urgently needed, the objective being to be able to screen out lung cancer patients with relatively poor prognosis and relatively fast cancer development, to give them more effective chemotherapy drugs in a timely manner, and to thereby save their lives.

Genomic imprinting is a gene regulation method in epigenetics and is characterized by modifying, or more specifically methylating, an allele from a specific parent such that only one allele of the corresponding gene is expressed while the other allele is in a silenced state. A gene regulated in this way is referred to as an imprinted gene. Loss of imprinting is an epigenetic change in which the silenced allele of an imprinted gene is demethylated and is thus activated and expressed. Numerous studies have shown that loss of imprinting exists widely in all kinds of cancers and takes place earlier than morphological changes in cells and tissue. Loss of imprinting, however, seldom occurs in healthy cells, which is in stark contrast to the case with cancer cells. Therefore, the methylated state of an imprinted gene can serve as a pathological marker and used in conjunction with specific molecular detection techniques to analyze cellular abnormality.

As the functions of an imprinted gene cover cell signaling, cell cycle regulation, substance transport across the cell membrane, the formation of extracellular matrix, and so on, the expressed functions and expressed quantity of an imprinted gene in a certain cancer may differ from those in another cancer, with the difference in expressed quantity being huge. An imprinted gene, therefore, may have different sensitivity and specificity to a certain cancer from another imprinted gene. The differences in sensitivity and specificity have a significant impact on the invasion and metastasis of tumor cells during tumor development and on the prognosis of tumors.

According to the above, typical diagnostic markers for lung cancer prognosis are currently unavailable. That is to say, there is presently no way to analyze changes in a molecular marker on a cellular level in order to provide more accurate prognosis and diagnosis information for lung cancer patients.

### BRIEF SUMMARY OF THE INVENTION

In view of the deficiency of the prior art and in order to meet practical needs, the present application provides a prognostic marker for lung cancer, a prognostic typing model for lung cancer, and applications thereof. The marker has a significant correlation with lung cancer prognosis. The prognostic typing model for lung cancer is created with the marker and is advantageous to the provision of accurate, useful prognosis information to lung cancer patients.

To achieve the foregoing objective, the following technical solution is used:

According to the first aspect of the present application, a prognostic marker for lung cancer is provided, and the marker includes imprinted gene Dcn.

In the present application, imprinted gene Dcn has a significant correlation with lung cancer prognosis. Even in cases where a lung cancer patient has received early treatment and the cancer is highly differentiated, a great product of a total expressed quantity of Dcn and an expressed quantity of Dcn with a copy number variation indicates that the patient has a poor prognosis, with a five-year survival rate lower than 10%. Imprinted gene Dcn is the most sensitive and most specific marker for lung cancer prognosis and can serve an indicative purpose earlier than clinicopathologic features.

Preferably, the marker further includes any one, or a combination of at least two, of imprinted genes Peg10, Snrpn/Snurf, and Trappc9.

Preferably, the marker includes imprinted genes Dcn, Peg10, Snrpn/Snurf, and Trappc9.

The four imprinted genes Dcn, Peg10, Snrpn/Snurf, and Trappc9 used in the present application have a significant correlation with lung cancer prognosis. The product of the total expressed quantity of each of the four imprinted genes and the expressed quantity of the imprinted gene with a copy number variation contributes to the creation of a prognostic typing model for lung cancer so as to predict the five-year survival rates of lung cancer patients individually and thereby provide accurate, useful prognosis information to lung cancer patients.

In the present application, a retrospective analysis was performed, via in-situ hybridization, on 155 paraffin-embedded lung cancer tissue samples with known five-year survival rate information. It was found that in lung cancer tissue samples whose corresponding five-year survival rates were lower than 10%, the product of the total expressed quantity of imprinted gene Dcn and the expressed quantity of imprinted gene Dcn with a copy number variation was not less than 1.5%;

that in lung cancer tissue samples whose corresponding five-year survival rates were lower than 25%, the product of the total expressed quantity of imprinted gene Dcn and the expressed quantity of imprinted gene Dcn with a copy number variation was less than 1.5%, the product of the total expressed quantity of imprinted gene Peg10 and the expressed quantity of imprinted gene Peg10 with a copy number variation was not less than 1%, and the product of the total expressed quantity of imprinted gene Snrpn/Snurf and the expressed quantity of imprinted gene Snrpn/Snurf with a copy number variation was not less than 1%;

that in lung cancer tissue samples whose corresponding five-year survival rates were lower than 35%, the product of the total expressed quantity of imprinted gene Dcn and the expressed quantity of imprinted gene Dcn with a copy number variation was less than 1.5%, the product of the total expressed quantity of imprinted gene Peg10 and the expressed quantity of imprinted gene Peg10 with a copy number variation was greater than 0 and less than 1% (or the product of the total expressed quantity of imprinted gene Snrpn/Snurf and the expressed quantity of imprinted gene Snrpn/Snurf with a copy number variation was greater than 0 and less than 1%), and the product of the total expressed quantity of imprinted gene Trappc9 and the expressed quantity of imprinted gene Trappc9 with a copy number variation was not less than 2%;

that in lung cancer tissue samples whose corresponding five-year survival rates were higher than 60%, the product of the total expressed quantity of imprinted gene Dcn and the expressed quantity of imprinted gene Dcn with a copy number variation was less than 1.5%, the product of the total expressed quantity of imprinted gene Peg10 and the expressed quantity of imprinted gene Peg10 with a copy number variation was greater than 0 and less than 1% (or the product of the total expressed quantity of imprinted gene Snrpn/Snurf and the expressed quantity of imprinted gene Snrpn/Snurf with a copy number variation was greater than 0 and less than 1%), and the product of the total expressed quantity of imprinted gene Trappc9 and the expressed quantity of imprinted gene Trappc9 with a copy number variation was less than 2%; and

that in lung cancer tissue samples whose corresponding five-year survival rates were 100%, the product of the total expressed quantity of imprinted gene Dcn and the expressed quantity of imprinted gene Dcn with a copy number variation was equal to 0, the product of the total expressed quantity of imprinted gene Peg10 and the expressed quantity of imprinted gene Peg10 with a copy number variation was equal to 0, and the product of the total expressed quantity of imprinted gene Snrpn/Snurf and the expressed quantity of imprinted gene Snrpn/Snurf with a copy number variation was equal to 0.

Preferably, the marker affects lung cancer prognosis through cancer-associated fibroblasts (CAF).

According to the second aspect of the present application, a prognostic typing model for lung cancer is provided, and the typing model uses the marker described in relation to the first aspect of the present application to carry out prognostic typing.

Preferably, the typing model includes type A, type B, type C, type D, and type E, in which:

type A: the product of the total expressed quantity of imprinted gene Dcn and the expressed quantity of imprinted gene Dcn with a copy number variation is not less than 1.5%;

type B: the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is not less than 1%, and the product of the total expressed quantity of imprinted gene Snrpn/Snurf and the expressed quantity of imprinted gene Snrpn/Snurf with a copy number variation is not less than 1%;

type C: the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is greater than 0 and less than 1% (or the product of the total expressed quantity of imprinted gene Snrpn/Snurf and the expressed quantity of imprinted gene Snrpn/Snurf with a copy number variation is greater than 0 and less than 1%), and the product of the total expressed quantity of imprinted gene Trappc9 and the expressed quantity of imprinted gene Trappc9 with a copy number variation is not less than 2%;

type D: the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is greater than 0 and less than 1% (or the product of the total expressed quantity of imprinted gene Snrpn/Snurf and the expressed quantity of imprinted gene Snrpn/Snurf with a copy number variation is greater than 0 and less than 1%), and the product of the total expressed quantity of imprinted gene Trappc9 and the expressed quantity of imprinted gene Trappc9 with a copy number variation is less than 2%; and

type E: the product of the total expressed quantity of imprinted gene Dcn and the expressed quantity of imprinted gene Dcn with a copy number variation is equal to 0, the product of the total expressed quantity of imprinted gene Peg10 and the expressed quantity of imprinted gene Peg10 with a copy number variation is equal to 0, and the product of the total expressed quantity of imprinted gene Snrpn/Snurf and the expressed quantity of imprinted gene Snrpn/Snurf with a copy number variation is equal to 0.

The five-year survival rate of type A is lower than 10%, the five-year survival rate of type B is 10%-25%, the five-year survival rate of type C is 25%-35%, the five-year survival rate of type D is higher than 60%, and the five-year survival rate of type E is 100%.

In the present application, the product of the total expressed quantity (TE) of an imprinted gene and the expressed quantity of the imprinted gene with a copy number variation (CNV) is used as a parameter of the typing model because according to the applicant's findings, most patients with a poor prognosis have relatively great TE and CNV values whereas some patients with a good prognosis have small TE values and great CNV values, meaning TE values or CNV values alone cannot clearly distinguish a good prognosis from a poor one. To increase the accuracy of the typing model, therefore, TExCNV is used as a parameter of the model.

Preferably, the following formulas are used to calculate the total expressed quantity of an imprinted gene and the expressed quantity of the imprinted gene with a copy number variation:

total expressed quantity of the imprinted gene=(b+c+d)/(a+b+c+d)×100%; and

expressed quantity of the imprinted gene with a copy number variation=d/(b+c+d)×100%;

where a is the number of cell nuclei that, after the corresponding cells are stained with hematoxylin, show no mark in each cell nucleus, meaning the imprinted gene is not expressed in those cell nuclei; b is the number of cell nuclei that, after the corresponding cells are stained with hematoxylin, show one red/brown mark in each cell nucleus, meaning the imprinted gene is present in those cell nuclei; c is the number of cell nuclei that, after the corresponding cells are stained with hematoxylin, show two red/brown marks in each cell nucleus, meaning the imprinted gene is affected by a loss of imprinting in those cell nuclei; and d is the number of cell nuclei that, after the corresponding cells are stained with hematoxylin, show more than two red/brown marks in each cell nucleus, meaning the imprinted gene shows a copy number variation in those cell nuclei.

According to the third aspect of the present application, a method for creating the typing model described in relation to the second aspect of the present application is provided, and the method includes the following steps:

(1) A probe for imprinted gene Dcn, Peg10, Snrpn/Snurf, or Trappc9 is used to perform in-situ hybridization on samples with known five-year survival rate information.

(2) a, b, c, and d are counted under a microscope. The total expressed quantity of the imprinted gene Peg10, Dcn, Snrpn/Snurf, or Trappc9 in each sample and the expressed quantity of the imprinted gene with a copy number variation in each sample are calculated according to the formulas with which to calculate those expressed quantities. The product of each total expressed quantity and the corresponding expressed quantity corresponding to a copy number variation is then calculated.

(3) A difference analysis is performed, by way of Student's t-test, on the products of the total expressed quantities of the imprinted gene and the expressed quantities of the imprinted gene with a copy number variation so as to create the typing model.

Preferably, the samples in step (1) include any one, or a combination of at least two, of paraffin-embedded lung cancer tissue samples, biopsy samples obtained through bronchoscopy, samples obtained by bronchial brushing, lung needle biopsy samples, samples obtained from a bronchoalveolar lavage fluid, samples obtained from a pleural fluid, and samples obtained from sputum.

In the present application, 155 paraffin-embedded lung cancer tissue samples were used, in which 49 samples had a good prognosis and 106 samples had a poor prognosis, the good-prognosis percentage being 31.6%, which is slightly higher than the average five-year survival rate (23%) of non-small-cell lung carcinoma (NSCLC). The good-prognosis percentage is generally consistent with the result of statistics from a large sample, ensuring that the model created does not deviate from actual conditions.

Preferably, the number of cells counted under the microscope in step (2) is 1000-3000 cells under a 400× objective lens/imprinted gene/sample.

In step (2), it is preferable that a is the number of cell nuclei that, after the corresponding cells are stained with hematoxylin, show no mark in each cell nucleus, meaning the imprinted gene is not expressed in those cell nuclei; that b is the number of cell nuclei that, after the corresponding cells are stained with hematoxylin, show one red/brown mark in each cell nucleus, meaning the imprinted gene is present in those cell nuclei; that c is the number of cell nuclei that, after the corresponding cells are stained with hematoxylin, show two red/brown marks in each cell nucleus, meaning the imprinted gene is affected by a loss of imprinting in those cell nuclei; and that d is the number of cell nuclei that, after the corresponding cells are stained with hematoxylin, show more than two red/brown marks in each cell nucleus, meaning the imprinted gene shows a copy number variation in those cell nuclei.

According to the fourth aspect of the present application, a prognostic typing method for lung cancer is provided. The typing method uses the typing model described in relation to the second aspect of the present application to determine the type of a sample taken from a lung cancer patient in order to predict the five-year survival rate of the lung cancer patient.

Preferably, the method includes the following steps:

(1′) A probe for imprinted gene Dcn, Peg10, Snrpn/Snurf, or Trappc9 is used to perform in-situ hybridization on a test sample.

(2′) a, b, c, and d are counted under a microscope. The total expressed quantity of the imprinted gene Peg10, Dcn, Snrpn/Snurf, or Trappc9 in the sample and the expressed quantity of the imprinted gene with a copy number variation in the sample are calculated according to the formulas with which to calculate those expressed quantities. The product of the total expressed quantity and the expressed quantity corresponding to a copy number variation is then calculated.

(3′) The type of the lung cancer patient's tissue sample is determined according to the typing model described in relation to the second aspect of the present application.

According to the fifth aspect of the present application, a use of the marker described in relation to the first aspect of the present application and/or the typing model described in relation to the second aspect of the present application in preparing a diagnostic reagent for predicting lung cancer prognosis and/or a therapeutic drug for improving lung cancer prognosis is provided.

According to the sixth aspect of the present application, a diagnostic reagent for predicting lung cancer prognosis is provided, and the diagnostic reagent includes a probe for detecting the marker described in relation to the first aspect of the present application.

Preferably, the probe is targeted at an intron of an imprinted gene serving as the marker.

According to the seventh aspect of the present application, a diagnostic reagent kit for predicting lung cancer prognosis is provided, and the reagent kit includes the diagnostic reagent described in relation to the sixth aspect of the present application.

Preferably, the reagent kit further includes an in-situ hybridization reagent.

Preferably, the in-situ hybridization reagent includes any one, or a combination of at least two, of xylene, hydrogen peroxide, a color-developing agent, and hematoxylin.

According to the eighth aspect of the present application, a therapeutic drug for improving lung cancer prognosis is provided, and the therapeutic drug includes the sgRNA of imprinted gene Dcn.

Preferably, the sgRNA includes the nucleic acid sequence of SEQ ID NO: 1 and/or the nucleic acid sequence of SEQ ID NO: 2:

SEQ ID NO: 1:
ATAAAATATGAAGCTGATCT;
SEQ ID NO: 2:
TAGTAAGGGCACTATTTCAT.

Preferably, the therapeutic drug further includes the Cas9 protein.

Preferably, the therapeutic drug further includes any one, or a combination of at least two, of a pharmaceutically acceptable carrier, a pharmaceutically acceptable excipient, and a pharmaceutically acceptable diluent.

Compared with the prior art, the present application has the following advantageous effects:

(1) The imprinted genes Dcn, Peg10, Snrpn/Snurf, and Trappc9 used in the present application have a significant correlation with lung cancer prognosis. In particular, a great product of the total expressed quantity of imprinted gene Dcn and the expressed quantity of imprinted gene Dcn with a copy number variation indicates that the lung cancer patient in question has a poor prognosis, with a five-year survival rate lower than 10%. Imprinted gene Dcn is the most sensitive and most specific marker for lung cancer prognosis and can serve an indicative purpose earlier than clinicopathologic features.

(2) According to the present application, the prognostic typing model for lung cancer is created based on the products of the total expressed quantities of four imprinted genes and the expressed quantities of those imprinted genes with a copy number variation and can accurately determine the type of prognosis of a lung cancer sample to help predict the five-year survival rate of an individual lung cancer patient, to provide guidance on the choice of medication, and to hopefully reduce the chance of post-operative recurrence and metastasis.

(3) According to an analysis in the present application, the expression of a copy number variation of imprinted gene Dcn may lead to a poor prognosis by way of CAF.

### BRIEF DESCRIPTION OF THE SEVERAL VIEWS OF THE DRAWINGS

FIG. 1 shows the expression of four imprinted genes in certain samples in the first embodiment of the present application, wherein some of the samples have a good prognosis and the others have a poor one;

FIG. 2 shows a comparison of the expression of four imprinted genes in the samples in the second embodiment of the present application, wherein some of the samples have a good prognosis and the others have a poor one;

FIG. 3 schematically shows the prognostic typing model in the third embodiment of the present application;

FIG. 4 shows the percentages of samples with a good/poor prognosis in the five types in the third embodiment of the present application; and

FIG. 5(A) shows the expression of CAF markers in samples with high Dcn expression and a poor prognosis, FIG. 5(B) shows the expression of the CAF markers in samples with no Dcn expression and a good prognosis, FIG. 5(C) shows the expression of the CAF markers in cell lines where gene Dcn is not knocked out, and FIG. 5(D) shows the expression of the CAF markers in cell lines where gene Dcn has been knocked out.

### DETAILED DESCRIPTION OF THE INVENTION

To further explain the technical solution used in the present application and its effects, certain embodiments are detailed below with reference to the accompanying drawings. It should be understood that the embodiments described herein serve only to expound, but not to limit the scope of, the subject matter of the present application.

While some of the techniques or conditions used in the embodiments may not be specified herein, those techniques or conditions can be implemented according to the corresponding description in literature in the same technical field as the present application or according to the corresponding product manuals. Any reagent or instrument that was used in the embodiments but whose manufacturer is not specified herein is a conventional product commercially available through regular marketing channels.

### Embodiment 1: Detection of Imprinted Genes in Samples

155 samples were studied in the present application. These samples have known five-year (2010-2014) survival rate information and are pathologically verified, formalin-fixed paraffin-embedded (FFPE) non-small-cell lung carcinoma (NSCLC) tissue samples.

In this embodiment, the samples in the training set received a retrospective analysis, in which in-situ hybridization was carried out between an in-situ hybridization probe for each of imprinted genes Dcn, Peg10, Snrpn/Snurf, and Trappc9 and the expression locus of the corresponding imprinted gene in the test samples. The analysis steps are as follows:

(1) The FFPE samples were cut into 10-μm sections, and the sections were placed on positively charged slides and allowed to dry overnight at room temperature.

(2) The samples were processed with the RNAscope 2.5 HD reagent kit (ACD) in the following manner. To begin with, the samples were dewaxed in xylene, and hydrogen peroxide was used to block the activity of the endogenous peroxidase in the samples. After that, the samples were incubated in a retrieval buffer for some time, and RNAscope Protease Plus Reagent (ACD) was used to enhance the permeability of the samples and expose the RNA molecules.

(3) In-situ hybridization was performed after the in-situ hybridization probes were designed according to the intron sequences of imprinted genes Dcn, Peg10, Snrpn/Snurf, and Trappc9. The probes were provided by Advanced Cell Diagnostics.

(4) The color-developing agent Fast Red (ACD) was added for signal amplification and detection. Then, the samples were stained with hematoxylin, and the expression of the imprinted genes was observed under a microscope.

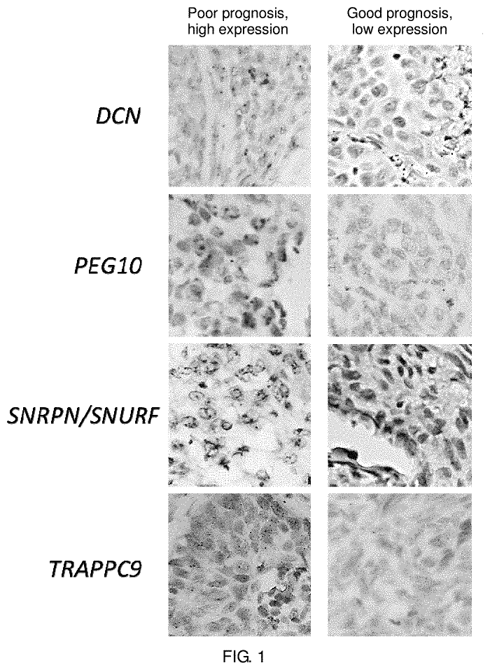

According to the analysis results as shown in FIG. 1, all of imprinted genes Dcn, Peg10, Snrpn/Snurf, and Trappc9 had relatively great total expressed quantities and showed large quantities of copy number variations in samples with a poor prognosis. In samples with a good prognosis, however, imprinted genes Dcn, Peg10, Snrpn/Snurf, and Trappc9 were basically not expressed.

### Embodiment 2: Data Collection and Statistical Analysis

The following were counted under a 400× objective lens: a, which is the number of cell nuclei in each of which no mark existed, meaning the imprinted gene to be detected was not expressed in those cell nuclei; b, which is the number of cell nuclei in each of which one red/brown mark existed, meaning the imprinted gene to be detected was present in those cell nuclei; c, which is the number of cell nuclei in each of which two red/brown marks existed, meaning the imprinted gene to be detected was affected by a loss of imprinting in those cell nuclei; and d, which is the number of cell nuclei in each of which more than two red/brown marks existed, meaning the imprinted gene to be detected showed a copy number variation in those cell nuclei. The number of cells counted under the 400× objective lens was 2000 cells/imprinted gene/sample.

The following formulas were used to calculate the total expressed quantity (TE) of an imprinted gene and the expressed quantity of the imprinted gene with a copy number variation (CNV), and a TE×CNV value was calculated accordingly:

total expressed quantity (TE) of the imprinted gene=(b+c+d)/(a+b+c+d)×100%; and

expressed quantity of the imprinted gene with a copy number variation (CNV)=d/(b+c+d)×100%.

Using Student's t-test, a difference analysis was performed on the TExCNV values of samples with a good prognosis and of samples with a poor prognosis.

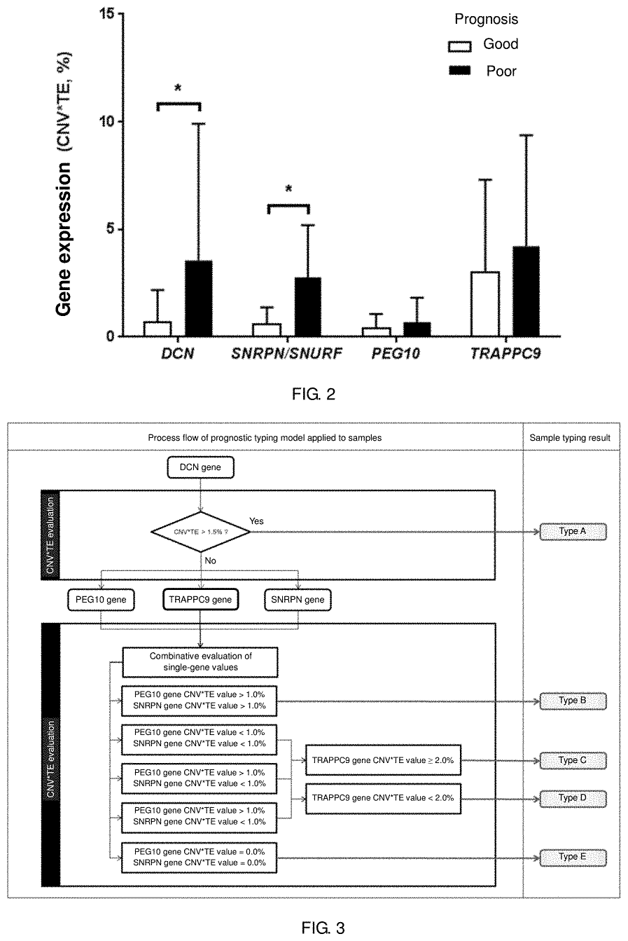

According to the analysis results as shown in FIG. 2, Dcn, Peg10, Snrpn/Snurf, and Trappc9 (in particular Dcn and Snrpn/Snurf) had significantly (p<0.05) greater expressed quantities in samples with a poor prognosis. Therefore, imprinted genes Dcn, Peg10, Snrpn/Snurf, and Trappc9 can serve as prognostic markers for lung cancer.

### Embodiment 3: Creation of a Typing Model

Based on the TE×CNV values of the four imprinted genes Dcn, Peg10, Snrpn/Snurf, and Trappc9, the types of the samples in the training set were determined, and a prognostic typing model for lung cancer was thus created, as shown in FIG. 3. The typing model includes five types, namely type A, type B, type C, type D, and type E:

type A (five-year survival rate being lower than 10%): the product of the total expressed quantity of imprinted gene Dcn and the expressed quantity of imprinted gene Dcn with a copy number variation is not less than 1.5%;

type B (five-year survival rate being 10%-25%): the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is not less than 1%, and the product of the total expressed quantity of imprinted gene Snrpn/Snurf and the expressed quantity of imprinted gene Snrpn/Snurf with a copy number variation is not less than 1%;

type C (five-year survival rate being 25%-35%): the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is greater than 0 and less than 1% (or the product of the total expressed quantity of imprinted gene Snrpn/Snurf and the expressed quantity of imprinted gene Snrpn/Snurf with a copy number variation is greater than 0 and less than 1%), and the product of the total expressed quantity of imprinted gene Trappc9 and the expressed quantity of imprinted gene Trappc9 with a copy number variation is not less than 2%;

type D (five-year survival rate being higher than 60%): the product of the total expressed quantity of imprinted gene Dcn and the expressed quantity of imprinted gene Dcn with a copy number variation is less than 1.5%, the product of the total expressed quantity of imprinted gene Peg10 and the expressed quantity of imprinted gene Peg10 with a copy number variation is greater than 0 and less than 1% (or the product of the total expressed quantity of imprinted gene Snrpn/Snurf and the expressed quantity of imprinted gene Snrpn/Snurf with a copy number variation is greater than 0 and less than 1%), and the product of the total expressed quantity of imprinted gene Trappc9 and the expressed quantity of imprinted gene Trappc9 with a copy number variation is less than 2%; and

type E (five-year survival rate being 100%): the product of the total expressed quantity of imprinted gene Dcn and the expressed quantity of imprinted gene Dcn with a copy number variation is equal to 0.

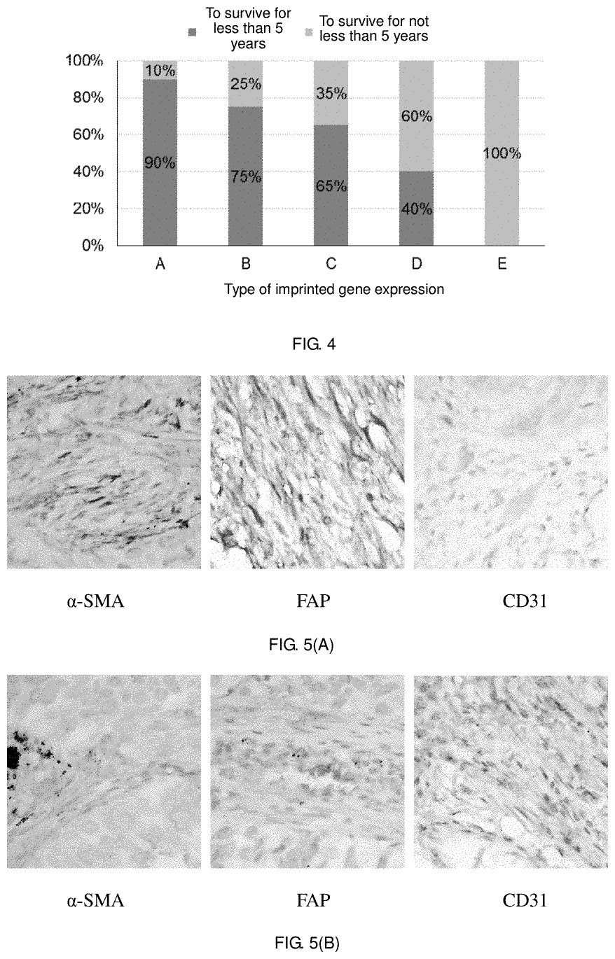

FIG. 4 shows the typing results of the 155 samples.

### Embodiment 4: Research into the Relationship Between Imprinted Gene Dcn and CAF

The samples in the training set in embodiment 1 were subjected to immunohistochemical staining immediately after in-situ hybridization.

The tissue sections were boiled in a sodium citrate solution for 1 hour and then blocked in 2% bovine serum albumin (BSA) for 30 minutes.

Cancer-associated fibroblast (CAF) markers α-SMA (α-smooth muscle actin) and FAP (fibroblast activation protein) were detected with rabbit anti-α-SMA antibody (Cell Signaling Technology) and rabbit anti-FAP antibody (Abcam) respectively. In addition, CD31, which is a negative marker for CAF, was detected with rabbit anti-CD31 (BBI life science).

Decorin, which is the expressed protein of imprinted gene Dcn, was detected with rabbit anti-decorin antibody (BBI life science).

All the antibodies were diluted with 2% BSA and then incubated with their respective intended samples overnight at 4° C. After washing with phosphate-buffered saline (PBS), the samples were added with HRP (horseradish peroxidase)-conjugated goat anti-rabbit secondary antibody (BBI life science), incubated at room temperature for 20 minutes, and then washed again with PBS.

The color-developing agent DAB (BBI life science) was added to facilitate signal detection, and the samples were observed under a microscope.

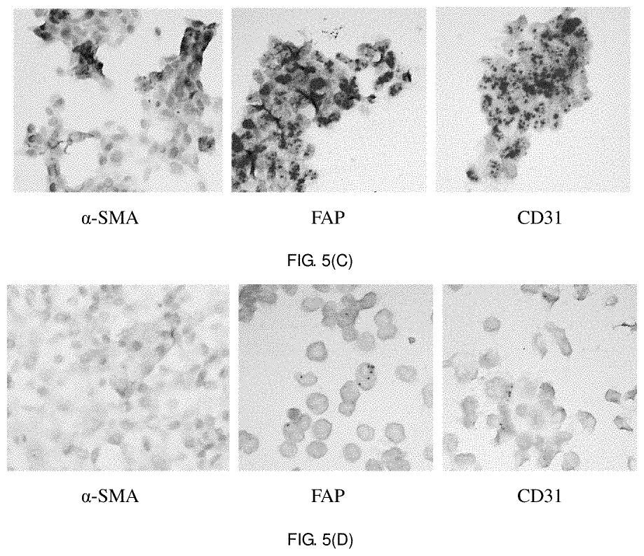

In type-A samples, as shown in FIG. 5(A), the CAF markers were highly expressed in matrix areas where Dcn was also highly expressed. In type-E samples, as shown in FIG. 5(B), Dcn was not expressed, and the CAF markers were expressed in very low quantities.

Using the CRIPSR (clustered regularly interspaced short palindromic repeats)/Cas9 technique, the imprinted gene Dcn in the mesenchymal stem cells HPM was knocked out with the imprinted gene Dcn-specific sgRNAs of SEQ ID NO: 1 and SEQ ID NO: 2. As a result, referring to FIG. 5(C) and FIG. 5(D), the expressed quantities of α-SMA and FAP in the HPM were significantly reduced.

It can be known from the above that the expression of a copy number variation of imprinted gene Dcn may result in a poor prognosis through CAF.

According to the foregoing, the imprinted genes Dcn, Peg10, Snrpn/Snurf, and Trappc9 used in the present application have a significant correlation with lung cancer prognosis. In particular, a great product of the total expressed quantity of imprinted gene Dcn and the expressed quantity of imprinted gene Dcn with a copy number variation indicates that the lung cancer patient in question has a poor prognosis, with a five-year survival rate lower than 10%. Imprinted gene Dcn is the most sensitive and most specific marker for lung cancer prognosis and can serve an indicative purpose earlier than clinicopathologic features. The prognostic typing model for lung cancer is created based on the products of the total expressed quantities of the four imprinted genes and the expressed quantities of those imprinted genes with a copy number variation and can accurately determine the type of prognosis of a lung cancer sample to help predict the five-year survival rate of an individual lung cancer patient and provide guidance on the choice of medication. An analysis in the present application also shows that the expression of a copy number variation of imprinted gene Dcn may lead to a poor prognosis by way of CAF.

The applicant would like to point out that, while the method of the present application has been described in detail by way of the foregoing embodiments, the present application is not limited to the method detailed above; in other words, implementation of the subject matter of the present application does not necessarily depend on the method detailed above. As would be understood by a person skilled in the art, any improvement made to the present application, any equivalent substitution of, and the addition of any auxiliary ingredient into, the raw materials used in the product of the present application, and any specific method chosen to implement the subject matter of the present application shall fall within the scope of the present application and of the patent protection sought by the applicant.

1. A prognostic marker for lung cancer, comprising imprinted gene Dcn.

2. The marker of claim 1, wherein the marker further comprises any one, or a combination of at least two, of imprinted genes Peg10, Snrpn/Snurf, and Trappc9.

3. The marker of claim 1, wherein the marker affects lung cancer prognosis through cancer-associated fibroblasts (CAF).

4. A prognostic typing model for lung cancer, wherein the typing model uses the marker of claim 1 to carry out prognostic typing.

5. The typing model of claim 4, wherein the typing model comprises type A, type B, type C, type D, and type E, wherein:
type A: a product of a total expressed quantity of the imprinted gene Dcn and an expressed quantity of the imprinted gene Dcn with a copy number variation is not less than 1.5%;
type B: the product of the total expressed quantity of the imprinted gene Dcn and the expressed quantity of the imprinted gene Dcn with a copy number variation is less than 1.5%, a product of a total expressed quantity of the imprinted gene Peg10 and an expressed quantity of the imprinted gene Peg10 with a copy number variation is not less than 1%, and a product of a total expressed quantity of the imprinted gene Snrpn/Snurf and an expressed quantity of the imprinted gene Snrpn/Snurf with a copy number variation is not less than 1%;
type C: the product of the total expressed quantity of the imprinted gene Dcn and the expressed quantity of the imprinted gene Dcn with a copy number variation is less than 1.5%, the product of the total expressed quantity of the imprinted gene Peg10 and the expressed quantity of the imprinted gene Peg10 with a copy number variation is greater than 0 and less than 1% (or the product of the total expressed quantity of the imprinted gene Snrpn/Snurf and the expressed quantity of the imprinted gene Snrpn/Snurf with a copy number variation is greater than 0 and less than 1%), and a product of a total expressed quantity of the imprinted gene Trappc9 and an expressed quantity of the imprinted gene Trappc9 with a copy number variation is not less than 2%;
type D: the product of the total expressed quantity of the imprinted gene Dcn and the expressed quantity of the imprinted gene Dcn with a copy number variation is less than 1.5%, the product of the total expressed quantity of the imprinted gene Peg10 and the expressed quantity of the imprinted gene Peg10 with a copy number variation is greater than 0 and less than 1% (or the product of the total expressed quantity of the imprinted gene Snrpn/Snurf and the expressed quantity of the imprinted gene Snrpn/Snurf with a copy number variation is greater than 0 and less than 1%), and the product of the total expressed quantity of the imprinted gene Trappc9 and the expressed quantity of the imprinted gene Trappc9 with a copy number variation is less than 2%; and
type E: the product of the total expressed quantity of the imprinted gene Dcn and the expressed quantity of the imprinted gene Dcn with a copy number variation is equal to 0, the product of the total expressed quantity of the imprinted gene Peg10 and the expressed quantity of the imprinted gene Peg10 with a copy number variation is equal to 0, and the product of the total expressed quantity of the imprinted gene Snrpn/Snurf and the expressed quantity of the imprinted gene Snrpn/Snurf with a copy number variation is equal to 0;
wherein type A has a five-year survival rate lower than 10%, type B has a five-year survival rate of 10%-25%, type C has a five-year survival rate of 25%-35%, type D has a five-year survival rate higher than 60%, and type E has a five-year survival rate of 100%.

6. The typing model of claim 4, wherein the total expressed quantity of each said imprinted gene and the expressed quantity of each said imprinted gene with a copy number variation are calculated using the following formulas:
the total expressed quantity of a said imprinted gene=(b+c+d)/(a+b+c+d)×100%; and
the expressed quantity of the imprinted gene with a copy number variation=d/(b+c+d)×100%;
where a is the number of cell nuclei that, after corresponding cells are stained with hematoxylin, show no mark in each said cell nucleus, meaning the imprinted gene is not expressed in those cell nuclei; b is the number of cell nuclei that, after corresponding cells are stained with hematoxylin, show one red/brown mark in each said cell nucleus, meaning the imprinted gene is present in those cell nuclei; c is the number of cell nuclei that, after corresponding cells are stained with hematoxylin, show two red/brown marks in each said cell nucleus, meaning the imprinted gene is affected by a loss of imprinting in those cell nuclei; and d is the number of cell nuclei that, after corresponding cells are stained with hematoxylin, show more than two red/brown marks in each said cell nucleus, meaning the imprinted gene shows a copy number variation in those cell nuclei.

7. A method for creating the typing model of claim 4, comprising the steps of:
(1) performing in-situ hybridization on samples with known five-year survival rate information, using a probe for the imprinted gene Dcn, of the imprinted gene Peg10, of (the) imprinted gene Snrpn/Snurf, or of the imprinted gene Trappc9;
(2) counting a, b, c, and d under a microscope; calculating a total expressed quantity of the imprinted gene Peg10, Dcn, Snrpn/Snurf, or Trappc9 in each said sample and an expressed quantity of the imprinted gene Peg10, Dcn, Snrpn/Snurf, or Trappc9 with a copy number variation in each said sample according to formulas with which to calculate said expressed quantities; and calculating a product of each said total expressed quantity and a corresponding said expressed quantity corresponding to a copy number variation; and
(3) performing a difference analysis, by way of Student's t-test, on the products of the total expressed quantities of the imprinted gene and the expressed quantities of the imprinted gene with a copy number variation so as to create the typing model.

8. The method of claim 7, wherein the samples in step (1) comprise any one, or a combination of at least two, of paraffin-embedded lung cancer tissue samples, biopsy samples obtained through bronchoscopy, samples obtained by bronchial brushing, lung needle biopsy samples, samples obtained from a bronchoalveolar lavage fluid, samples obtained from a pleural fluid, and samples obtained from sputum;
the number of cells counted under the microscope in step (2) is preferably 1000-3000 cells under a 400× objective lens/imprinted gene/sample; and
it is preferable in step (2) that a is the number of cell nuclei that, after corresponding cells are stained with hematoxylin, show no mark in each said cell nucleus, meaning the imprinted gene is not expressed in those cell nuclei; that b is the number of cell nuclei that, after corresponding cells are stained with hematoxylin, show one red/brown mark in each said cell nucleus, meaning the imprinted gene is present in those cell nuclei; that c is the number of cell nuclei that, after corresponding cells are stained with hematoxylin, show two red/brown marks in each said cell nucleus, meaning the imprinted gene is affected by a loss of imprinting in those cell nuclei; and that d is the number of cell nuclei that, after corresponding cells are stained with hematoxylin, show more than two red/brown marks in each said cell nucleus, meaning the imprinted gene shows a copy number variation in those cell nuclei.

9. A use of the marker of claim 1 in preparing a diagnostic reagent for predicting lung cancer prognosis and/or a therapeutic drug for improving lung cancer prognosis.

10. A diagnostic reagent for predicting lung cancer prognosis, comprising a probe for detecting the marker of claim 1.

11. The reagent of claim 10, wherein the probe is targeted at an intron of a said imprinted gene serving as the marker.

12. A diagnostic reagent kit for predicting lung cancer prognosis, comprising the diagnostic reagent of claim 10.

13. The reagent kit of claim 12, wherein the reagent kit comprises an in-situ hybridization reagent; and
the in-situ hybridization reagent preferably comprises any one, or a combination of at least two, of xylene, hydrogen peroxide, a color-developing agent, and hematoxylin.

14. A therapeutic drug for improving lung cancer prognosis, comprising the sgRNA of imprinted gene Dcn.

15. The drug of claim 14, wherein the sgRNA comprises the nucleic acid sequence of SEQ ID NO: 1 and/or the nucleic acid sequence of SEQ ID NO: 2;
the therapeutic drug preferably further comprises Cas9 protein; and
the therapeutic drug preferably further comprises any one, or a combination of at least two, of a pharmaceutically acceptable carrier, a pharmaceutically acceptable excipient, and a pharmaceutically acceptable diluent.


SEQUENCE LISTING (2 sequences)

SEQ ID NO: 1 - DNA, 20 bp, Artificial Sequence
  sgRNA
ataaaatatgaagctgatct

SEQ ID NO: 2 - DNA, 20 bp, Artificial Sequence
  sgRNA
tagtaagggcactatttcat